(19) United States
(12) Patent Application Publication
(10) Pub. No.: US 2008/0206845 A1
(43) Pub. Date: Aug. 28, 2008

(54) Bioreactor analysis system

(76) Inventor: Emilio Barbera-Guillem, Powell, OH (US)

Correspondence Address: GALLAGHER & DAWSEY CO., L.P.A., P.O. BOX 785, COLUMBUS, OH, 43216, US

(21) Appl. No.: 11/709,946

(22) Filed: Feb. 23, 2007

(51) Int. Cl. C12M 1/36; C12M 1/38; C12M 3/00

(52) U.S. Cl. 435/286.2; 435/286.5; 435/287.1; 435/303.1

(57) ABSTRACT

A bioreactor analysis system for incubating and analysis of a bioreactive material. The system comprises at least one bioreactor, preferably controlled environment bioreactors. The bioreactor may be held in a sleeve, and multiple sleeves may form a series that moving bioreactors into various storage and interventional positions. At least one interventional assembly interacts with the bioreactor while in the sleeve, and alternately, additional interventional assemblies may interact with the bioreactor while out of the sleeve. A jacket with an access port may surround the bioreactor, which may include a temperature management system. Alternately, a plurality of bioreactors may be joined to a storage array by the cooperation of intrinsic structures in the bioreactors and array. A control system allows for multiple individualized commands to be directed to any one or many of the bioreactors, and may utilize programs resident in the system or in remote locations far from the system.

### TECHNICAL FIELD

The instant invention relates to a bioreactor analysis system for incubating and analysis of a bioreactive material, and in particular for the incubation and analysis of cultured cells.

### BACKGROUND OF THE INVENTION

Bioreactors are common laboratory and industrial installations used in the areas of cell culture, chemical production, fermentation, testing and analysis, and other biological processes well known to those skilled in the art. A bioreactor, as that term is used in this application, means any vessel capable of holding a bioreactive material. This may commonly include, by way of example only, cells in a cell culture medium.

In a traditional laboratory practice, bioreactive materials such as cells in a cell culture medium, are introduced into a bioreactor or vessel, which may include such well known small pieces of laboratory equipment as flasks, Petri dishes, or multi-well plates. The bioreactors or vessels are then placed in a biochamber in which such environmental parameters as temperature, humidity, and ambient gas composition are controlled. At various time and situations, the bioreactors or vessels receive interventions, which may include but are not limited to the injection or withdrawal of materials, assessment of various parameters such a pH, or concentration, such as by centrifugation, of the bioreactive materials. Traditionally, these interventions have involved labor-intensive repetitive interventions by human laboratory personnel or by automated systems. Numerous problems have plagued such interventions and have slowed the development of high through-put systems. These problems, addressed in part by the instant invention, have included the need to remove bioreactors from a biochamber for intervention, or the enclosing of intervention instruments within a biochamber where conditions may be highly unfavorable to such instruments. Many current automated systems also suffer from the need to move individual bioreactors to and from each step of various interventions to access various instruments, thereby complicating and slowing processes.

### SUMMARY OF INVENTION

In its most general configuration, the present invention advances the state of the art with a variety of new capabilities and overcomes many of the shortcomings of prior devices in new and novel ways. In its most general sense, the present invention overcomes the shortcomings and limitations of the prior art in any of a number of generally effective configurations.

In one configuration, the present invention relates to a bioreactor analysis system for incubating and analysis of a bioreactive material, comprising a controlled environment bioreactor. The term controlled environment bioreactor, as used herein, means a bioreactor that is able to self regulate at least one environmental parameter such as humidity conservation or gas exchange. By way of example and not limitation, this includes the controlled environmental bioreactor seen in copending U.S. patent application Ser. No. 11/056,725, which is capable of selectively controlling molecular diffusion between an atmosphere and the contents of the bioreactor.

The controlled environment bioreactor has a chamber for containing bioreactive material and the bioreactor is releasably held within a slot in a sleeve. The slot and sleeve are designed so that an exposed portion of the controlled environment bioreactor projects from the sleeve.

There is an instrumentation system with a mobile interventional assembly directed by a drive assembly to positions the mobile interventional assembly in proximity to the exposed portion of the controlled environment bioreactor projecting from the sleeve. This allows the mobile interventional assembly to intervene with the bioreactive material contained within the chamber while the controlled environment bioreactor remains in the sleeve.

In another embodiment, the system may further includes a manipulator capable of releasably coupling to the controlled environment bioreactor, moving it outside of the sleeve, and transporting it to a fixed interventional assembly. Thus, the fixed interventional assembly intervenes with the bioreactive material contained within the chamber while the controlled environment bioreactor is outside the sleeve.

One skilled in the art will appreciate that a plurality of sleeves may be employed advantageously. These may be deployed such that the sleeves may be arranged to form a connected sleeve series having a storage region and a presentation region, and the sleeves may typically have different spatial arrangements in the storage region and the presentation region. Again typically, the sleeves may be further apart, at least in part, in the presentation region.

In one embodiment the storage region has a supine section and a prone section, separated by the presentation region and the bioreactor exterior surface has a front and a back, such that when the controlled environment bioreactor is in the supine section, the bioreactor is oriented so that gravity pulls the bioreactive material toward a back of the bioreactor. Alternately, when the controlled environment bioreactor is in the prone section, the bioreactor is oriented so that gravity pulls the bioreactive material toward the front. The sleeve series is movable through the supine section, the presentation region, and the prone section. This allows the contents of the controlled environment bioreactor to agitate, and such movement tends to reverse the direction of gravitational force on any contents within the bioreactor.

In one embodiment, the bioreactor analysis system has a jacket with an access port that connects the jacket interior surface with the jacket exterior surface. Various temperature management systems may heat or cool the jacket and/or the sleeve series contained therein.

An embodiment of the bioreactor analysis system may use means other than sleeves to contain one or more of the controlled environment bioreactors, such as means formed with the bioreactor that releasably couple to a storage array. In alternative embodiments using traditional bioreactors such as flask, Petri dishes, multi-well plates or the like, the system may be enclosed within a biochamber, providing controlled environmental conditions such as temperature, humidity, atmospheric pressure, and ambient fluid composition.

Any of the previously described embodiments of the bioreactor analysis system (50) may further include a control system (2000) to control the operation of the sleeve drive (440) and the instrumentation system (800). The control system (2000) may include a first remote data input device (2100), a second remote data input device (2200), and a local data receiving device (2300). The local data receiving device (2300) is in operative communication with the sleeve drive (440), the instrumentation system (800), the first remote data input device (2100), and the second remote data input device (2200). The term “operative communication” includes, but is not limited to, wired and wireless data communication as would be known to one skilled in the art.

The instant invention is unique in the wide array of electronic control systems that may be used to control the system. The various functionalities of the system, such as environment conditions within the system, movement of the sleeves, and interventions between the bioreactors and various instrumentalities may be controlled by programs resident within the system, or preferably, by programs resident outside of the system, even far outside of the system. Furthermore, the system has great flexibility. For example, commands can be sent targeting specific individual bioreactors from computers, connected by example by the internet, from the farthest reaches of the world. Additionally, the system can operate multiple experimental protocols simultaneously, at the direction of multiple outside operators.

### BRIEF DESCRIPTION OF THE DRAWINGS

Without limiting the scope of the present invention as claimed below and referring now to the drawings and figures:

FIG. 1 shows a perspective view of an embodiment of the instant invention, not to scale;

FIG. 2 shows a perspective view of a detail of the sleeve and bioreactor of one embodiment of FIG. 1, not to scale;

FIG. 3 is a section view of a bioreactor of an embodiment of one embodiment of FIG. 2, taken along section line 3-3, not to scale;

FIG. 4 is a section view of a sleeve of an embodiment of FIG. 2, taken along section line 4-4, not to scale;

FIG. 5 is a perspective view of a sleeve of an embodiment of FIG. 1, not to scale;

FIG. 6 is a top plan view of a sleeve of an embodiment of FIG. 1, not to scale;

FIG. 7 is a rear elevation view of a sleeve of an embodiment of FIG. 1, not to scale;

FIG. 8 is a side elevation view of a sleeve of an embodiment of FIG. 1, not to scale;

FIG. 9 is a perspective view of a sleeve series of an embodiment of FIG. 1, not to scale;

FIG. 10 is a side schematic view of a sleeve series of an embodiment of FIG. 1, not to scale, with some sleeves removed for clarity;

FIG. 11 is a perspective view of a sleeve series and jacket of an embodiment of FIG. 1, not to scale;

FIG. 12 is another perspective view of a sleeve series and jacket of an embodiment of FIG. 1, not to scale;

FIG. 13 is a schematic view, of a jacket and incubation chamber temperature management system;

FIG. 14 is a perspective view of mobile interventional assemblies of an embodiment of FIG. 1, not to scale;

FIG. 15 is perspective view of a fixed interventional assembly of an embodiment of FIG. 1, not to scale;

FIG. 16 is a schematic view of a sleeve series, mobile interventional assemblies, and a manipulator assembly of an embodiment of FIG. 1, not to scale, with some sleeves removed for clarity;

FIG. 17 is an alternative embodiment of the invention of FIG. 1, wherein the system is shown enclosed in a biochamber;

FIG. 18 is a schematic view of a storage array and bioreactors of an alternate embodiment of the instant invention;

FIG. 19 is another schematic view of a storage array and bioreactors of an alternate embodiment of the instant invention; and

FIG. 20 is a schematic diagram of an embodiment of a control system for controlling the instant invention.

### DETAILED DESCRIPTION OF THE INVENTION

The method and materials of the bioreactor analysis system of the instant invention enables a significant advance in the state of the art. The preferred embodiments of the method and materials accomplish this by new and novel arrangements of elements and methods that are configured in unique and novel ways and which demonstrate previously unavailable but preferred and desirable capabilities.

The detailed description set forth below in connection with the drawings is intended merely as a description of the presently preferred embodiments of the invention, and is not intended to represent the only form in which the present invention may be constructed or utilized. The description sets forth the designs, functions, means, and methods of implementing the invention in connection with the illustrated embodiments. It is to be understood, however, that the same or equivalent functions and features may be accomplished by different embodiments that are also intended to be encompassed within the spirit and scope of the invention.

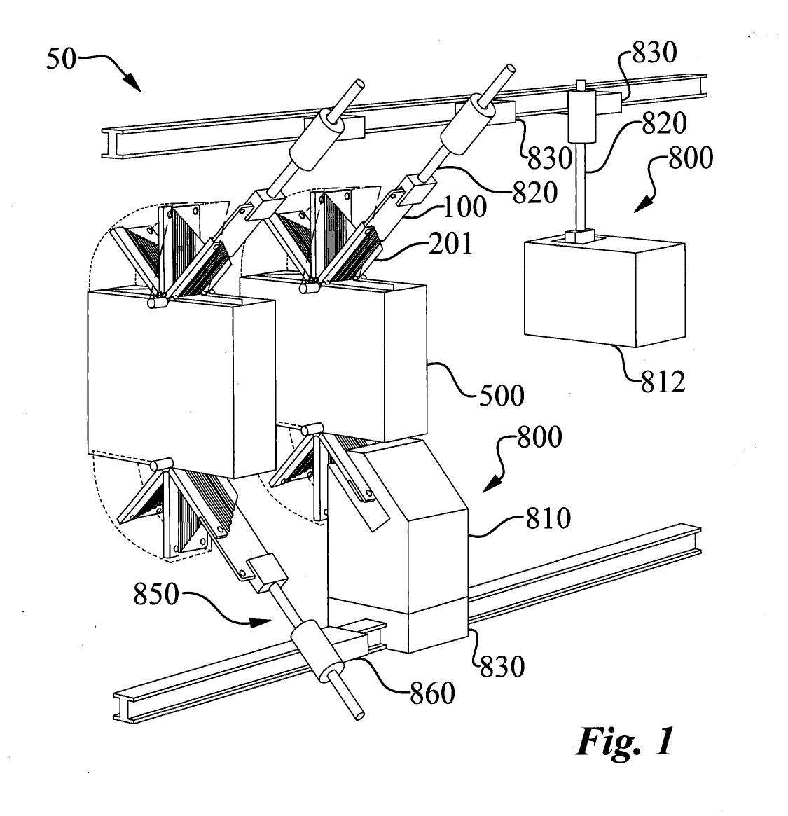

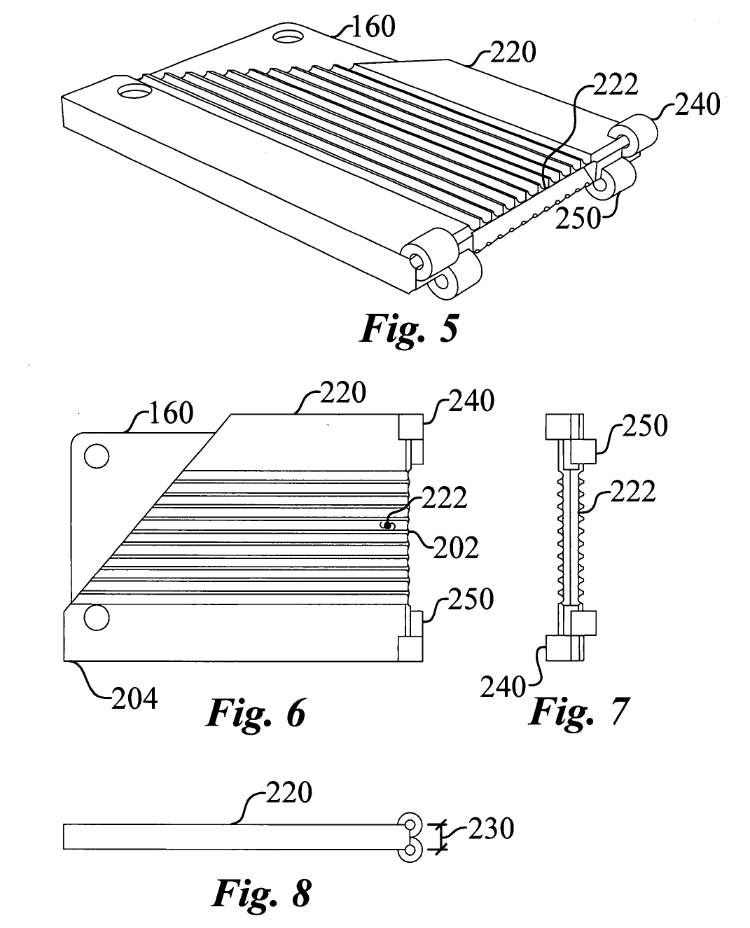

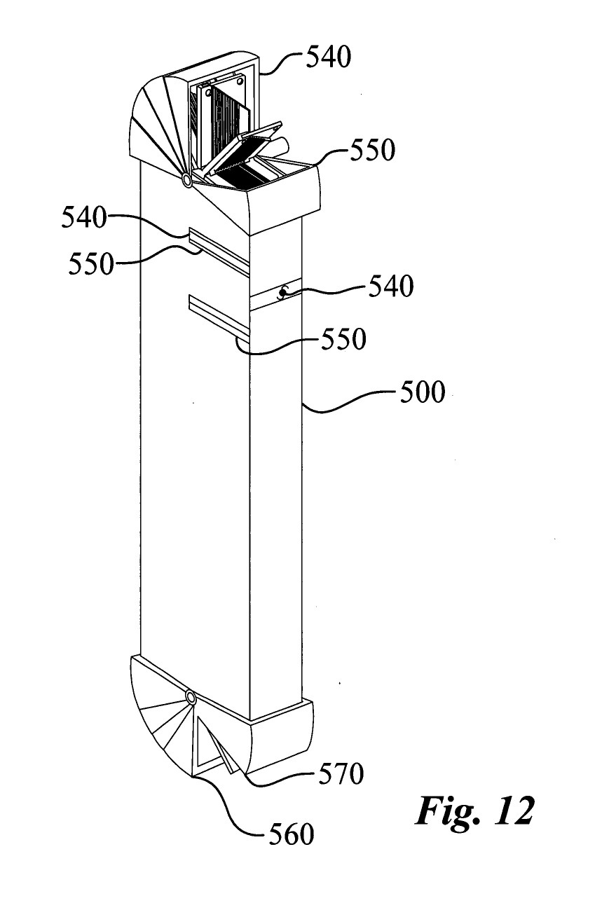

In a preferred embodiment, seen in FIGS. 1-20, the instant invention includes a bioreactor analysis system (50) for incubating and analysis of a bioreactive material, comprising a controlled environment bioreactor (100). The term controlled environment bioreactor, as used herein, means a bioreactor that is able to self regulate at least one environmental parameter such as humidity conservation or gas exchange. By way of example and not limitation, this includes the controlled environmental bioreactor seen in copending U.S. patent application Ser. No. 11/056,725, which is capable of selectively controlling molecular diffusion between an atmosphere and the contents of the bioreactor.

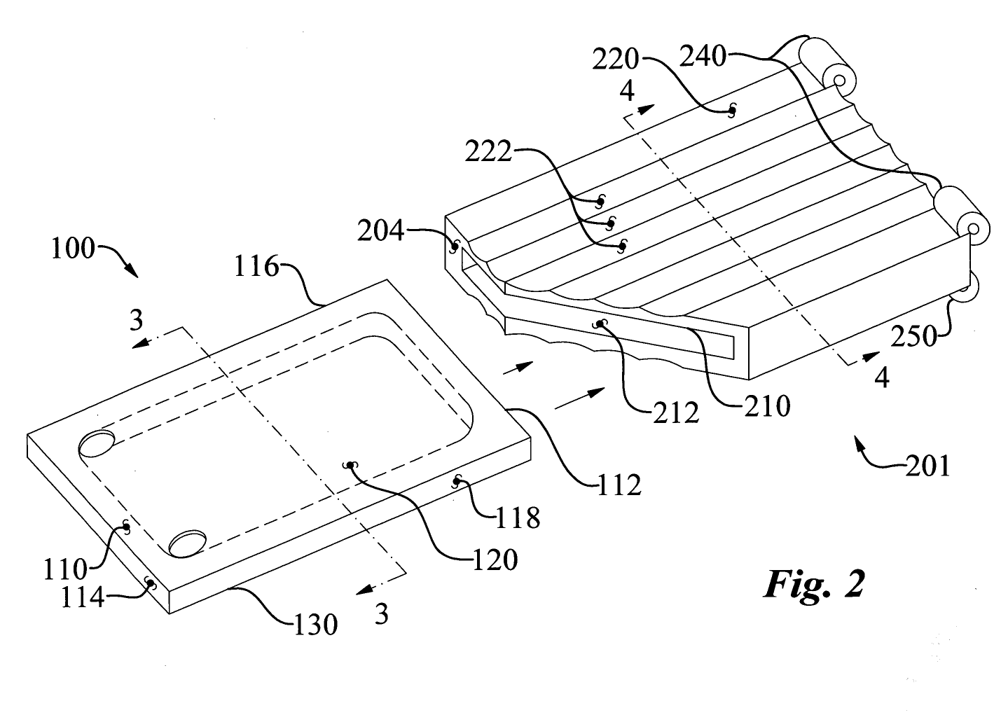

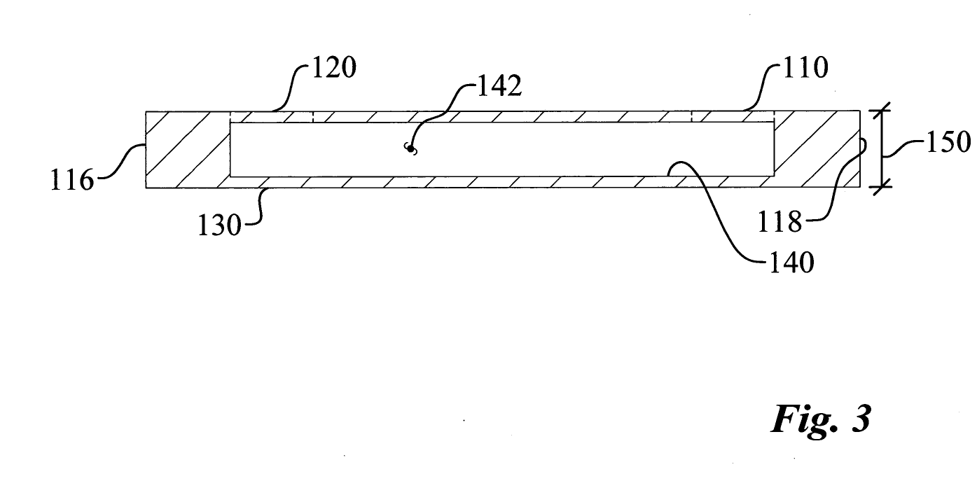

As seen in FIGS. 2 and 3, the controlled environment bioreactor has a bioreactor exterior surface (110), a bioreactor thickness (150), and a bioreactor interior surface (140). The bioreactor interior surface (140) forms a chamber (142) for containing the bioreactive material.

The bioreactor analysis system (50) also has a sleeve (201), seen best in FIGS. 2, 4, and 8, having a sleeve interior surface (210) and a sleeve exterior surface (220), for releasably holding the controlled environment bioreactor (100). The sleeve interior surface (210) forms a slot (212) that slidably receives the controlled environment bioreactor (100) such that an exposed portion (160) of the controlled environment bioreactor (100) projects from the sleeve (201). The sleeve embodiments shown in the figures illustrate a chamfered corner which thereby exposes a portion of the controlled environment bioreactor (100), but one skilled in the art will appreciate that the sleeve (201) may be configured in a number of different ways that also cause a portion of the controlled environment bioreactor (100) to remain exposed. For instance, in one simple example the length of the sleeve (201) is simply shorter than the length of the controlled environment bioreactor (100) thereby causing a portion of the controlled environment bioreactor (100) to always extend out of the sleeve (201).

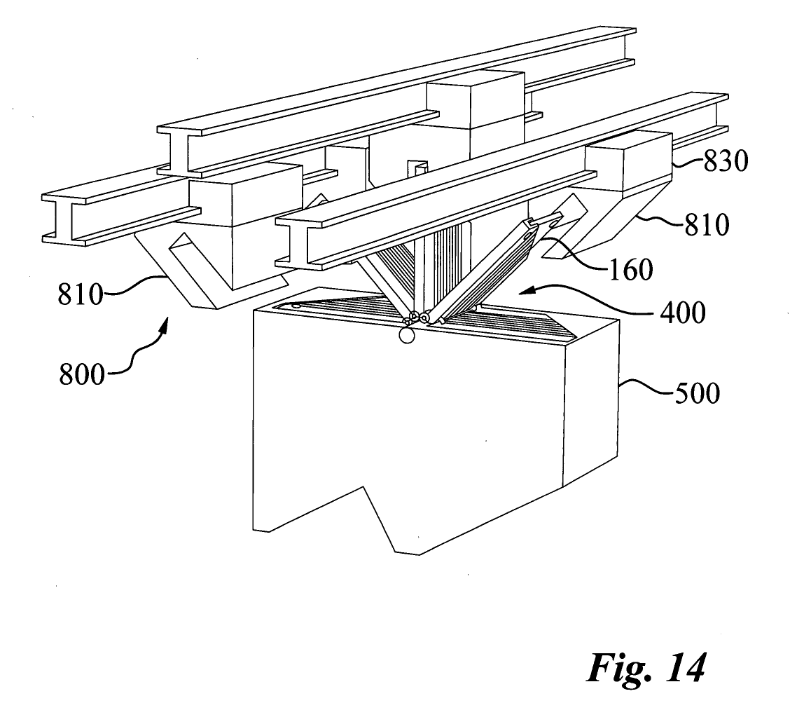

There is an instrumentation system (800) having a mobile interventional assembly (810) for intervening with the bioreactive material, seen in FIGS. 1 and 14. The instrumentation system (800) has a drive assembly (830) for moving the mobile interventional assembly (810), and the drive assembly (830) positions the mobile interventional assembly (810) in interventional proximity to the exposed portion (160) of the controlled environment bioreactor (100) that projects from the sleeve (201). This allows the mobile interventional assembly (810) to intervene with the bioreactive material contained within the chamber (142) while the controlled environment bioreactor (100) remains in the sleeve (201). As used herein interventional proximity means that the mobile interventional assembly (810) is brought close enough to the bioreactor exposed portion (160) to obtain the data that it is directed to obtain. Therefore, one skilled in the art will appreciate that some tasks, such as microscopic examination, may only require the mobile interventional assembly (810) to be within a few inches of the bioreactor exposed portion (160), while other operations that require sampling of the bioreactive material will require that the mobile interventional assembly (810) is in contact with a portion of the bioreactor exposed portion (160).

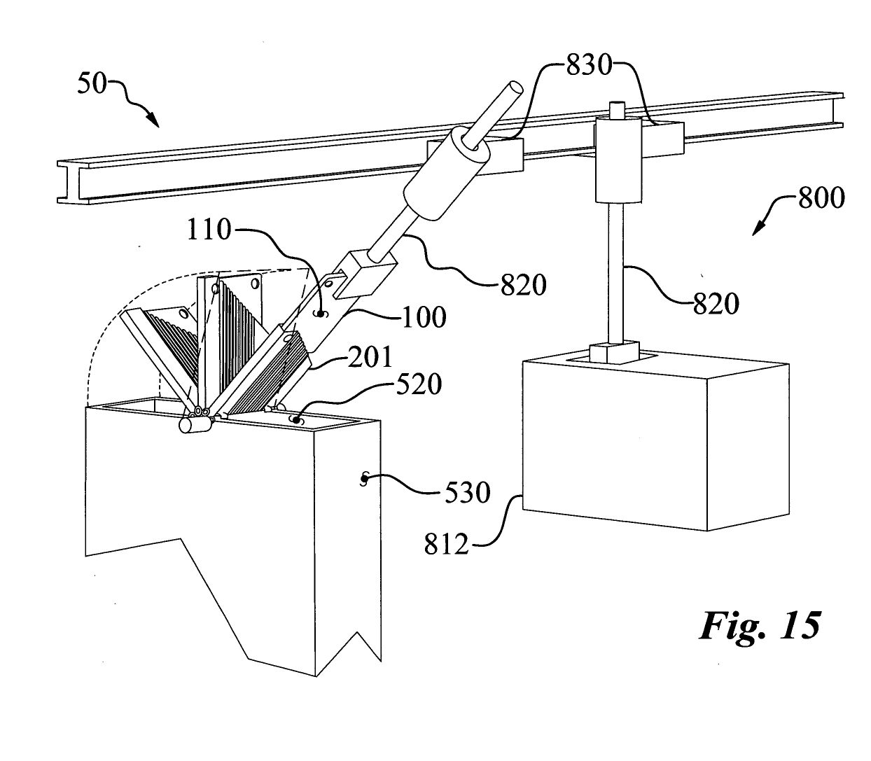

In another embodiment, seen best in FIGS. 1 and 15, the bioreactor analysis system (50) has an instrumentation system (800) that further includes a manipulator assembly (820) for intervening with the exposed portion (160) of the controlled environment bioreactor (100) projecting from the sleeve (201). The drive assembly (830) positions the manipulator assembly (820) in releasable coupling proximity to the controlled environment bioreactor (100) when the controlled environment bioreactor (100) is in the sleeve (201). This allows the manipulator assembly (820) to releasably couple to the controlled environment bioreactor (100), move the controlled environment bioreactor (100) outside of the sleeve (201), and transport the controlled element bioreactor (100) to the fixed interventional assembly (812). Thus, the fixed interventional assembly (812) intervenes with the bioreactive material contained within the chamber (142) while the controlled environment bioreactor (100) is outside the sleeve (201).

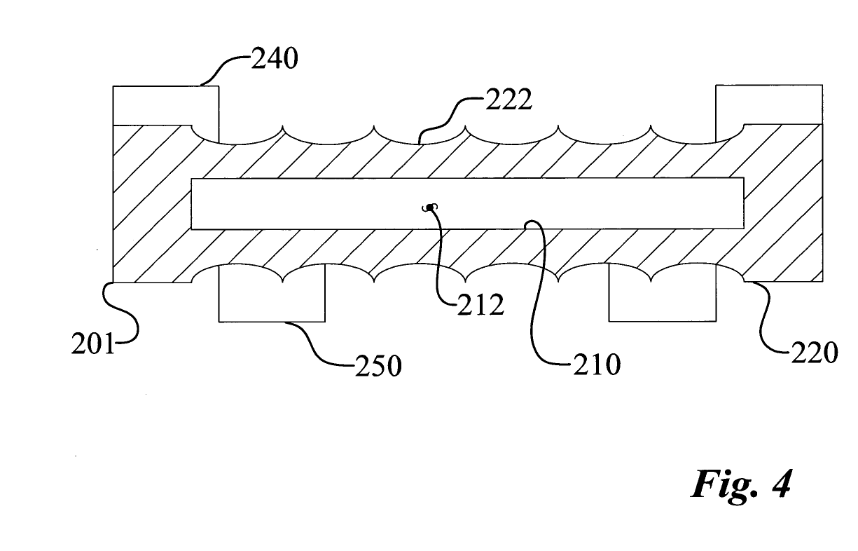

In one embodiment, the sleeve exterior surface (220) further includes at least one thermal exchange channel (222), seen in FIGS. 2 and 4, enhancing convective heat transfer between the sleeve (201) and an adjacent fluid. By way of example and not limitation, these channels enhance the heat exchange produced by the flow of warm or cool fluid across the sleeve.

One skilled in the art will appreciate that a plurality of sleeves (201) may be employed advantageously, seen well in FIGS. 1, 9, 10, 11, and 15-19. In yet another embodiment, the bioreactor analysis system may have at least a second sleeve (260) adjacent to the sleeve (201) with the second sleeve (260) having a second sleeve exterior surface (280), as well as a third sleeve (320) adjacent to the second sleeve (260), with the third sleeve (320) having a third sleeve exterior surface (340).

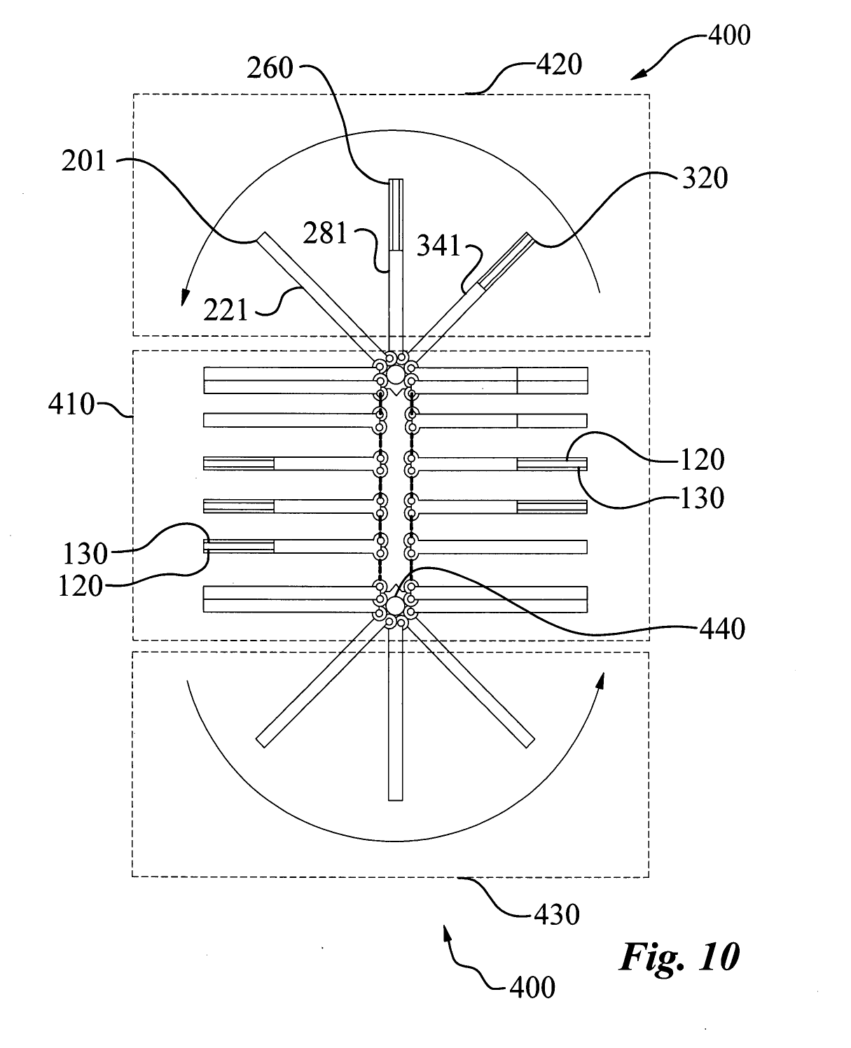

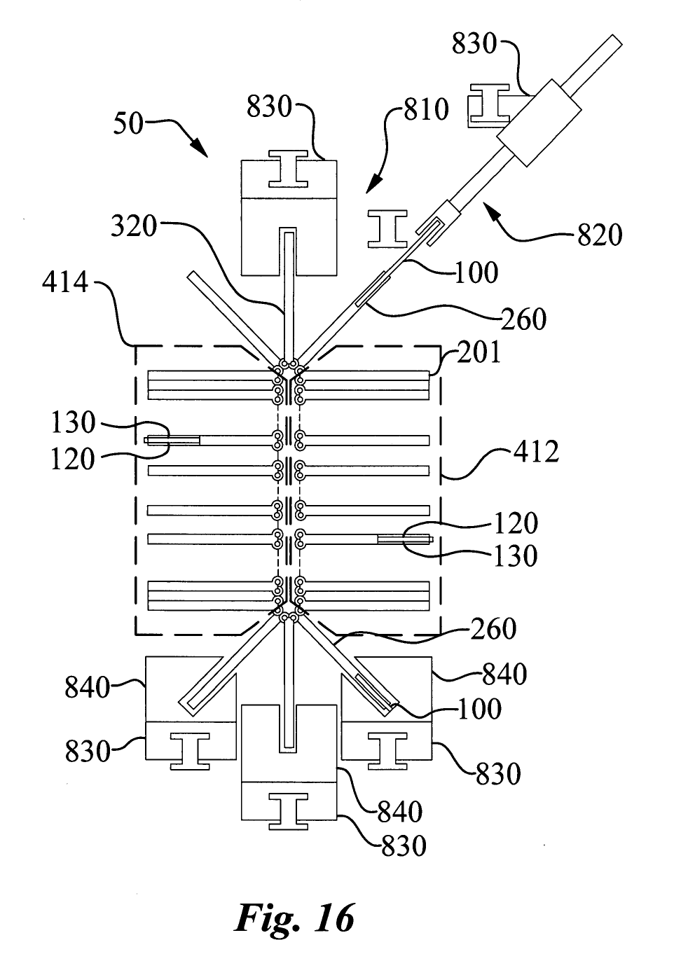

These may be deployed such that the sleeve (201), the second sleeve (260), and the third sleeve (320) are arranged to form a connected sleeve series (400) having a storage region (410) and a presentation region (420), as seen in FIGS. 10 and 16. In one embodiment, seen best in FIG. 10, in the presentation region (420) a primary sleeve exterior surface (221) of the sleeve (201) is not parallel to a primary sleeve exterior surface (281) of the second sleeve (260) and is not parallel to a primary exterior surface (341) of the third sleeve (320). To move the sleeve series (400), a sleeve drive (440) may be positioned to move the sleeve series (400) through the storage region (410) and the presentation region (420). The mobile interventional assembly (810) may access the controlled environment bioreactor (100) while the controlled environment bioreactor (100) is in the presentation region (420), as this would typically be the area where adjoining exposed portions (160) of controlled environment bioreactors (100) have the greatest separation.

Various spatial arrangements of the sleeve (201), the second sleeve (260) and the third sleeve (320) are possible in the storage region, seen in FIG. 10. In one embodiment, when in the storage region (410), the sleeve (201), the second sleeve (260), and the third sleeve (320) are positioned such that no portion of the sleeve external surface (220) is more distant from any portion of the second sleeve external surface (280) than ten times the bioreactor thickness (150), and no portion of the third sleeve external surface (340) is more distant from any portion of the second sleeve external surface (280) than ten times the bioreactor thickness (150).

In another embodiment, when in the storage region (410), the sleeve (201), the second sleeve (260), and the third sleeve (320) are positioned such that no portion of the sleeve external surface (220) is more distant from any portion of the second sleeve external surface (280) than five times the bioreactor thickness (150), and no portion of the third sleeve external surface (340) is more distant from any portion of the second sleeve external surface (280) than five times the bioreactor thickness (150).

In yet another embodiment, when in the storage region (410), the sleeve (201), the second sleeve (260), and the third sleeve (320) are positioned such that no portion of the sleeve external surface (220) is more distant from any portion of the second sleeve external surface (280) than two times the bioreactor thickness (150), and no portion of the third sleeve external surface (340) is more distant from any portion of the second sleeve external surface (280) than two times the bioreactor thickness (150). Further, in another embodiment a portion of the exterior surfaces of adjacent sleeves is in contact with the adjacent sleeve exterior surface thereby further maximizing the number of sleeves, and therefore bioreactors, that may be stored in a given volume.

Similarly, various spatial arrangements of the sleeve (201), the second sleeve (260) and the third sleeve (320) are possible in the presentation region, again well seen in FIG. 10. In one embodiment, when in the presentation region (420), the sleeve (201), the second sleeve (260), and the third sleeve (320) are positioned such that at least one portion of the sleeve external surface (220) is more distant from the nearest portion of the second sleeve external surface (280) than two times the bioreactor thickness (150), and at least one portion of the third sleeve external surface (340) is more distant from the nearest portion of the second sleeve external surface (280) than two times the bioreactor thickness (150). The spreading out of the sleeves in the presentation region (420) facilitates the cooperation and operation of the instrumentation assembly (800) without the need to remove the bioreactor (100).

In another embodiment, when in the presentation region (420), the sleeve (201), the second sleeve (260), and the third sleeve (320) are positioned such that at least one portion of the sleeve external surface (220) is more distant from the nearest portion of the second sleeve external surface (280) than five times the bioreactor thickness (150), and at least one portion of the third sleeve external surface (340) is more distant from the nearest portion of the second sleeve external surface (280) than five times the bioreactor thickness (150).

In yet another embodiment, when in the presentation region (420), the sleeve (201), the second sleeve (260), and the third sleeve (320) are positioned such that at least one portion of the sleeve external surface (220) is more distant from the nearest portion of the second sleeve external surface (280) than ten times the bioreactor thickness (150), and at least one portion of the third sleeve external surface (340) is more distant from the nearest portion of the second sleeve external surface (280) than ten times the bioreactor thickness (150).

In a preferred embodiment, seen well in FIGS. 1, 9, 10, 11, and 15-19, when in the presentation region (420), the sleeve (201), the second sleeve (260), and the third sleeve (320) all have primary external surfaces (221, 281, 341) which are non-parallel. In one embodiment, when in the presentation region (420), the sleeve (201), the second sleeve (260), and the third sleeve (320) are positioned such that at least one portion of the sleeve external surface (220) is more distant from the nearest portion of the second sleeve external surface (280) than ten times the bioreactor thickness (150) and at least one portion of the sleeve external surface (220) is less distant from the nearest portion of the second sleeve external surface (280) than ten times the bioreactor thickness, and at least one portion of the third sleeve external surface (340) is more distant from the nearest portion of the second sleeve external surface (280) than ten times the bioreactor thickness (150), and at least one portion of the third sleeve external surface (340) is less distant from the nearest portion of the sleeve external surface (280) than ten times the bioreactor thickness (150). Thus, the sleeves tend to fan out to cooperate with the instrumentation system (800).

In another embodiment, when in the presentation region (420), the sleeve (201), the second sleeve (260), and the third sleeve (320) are positioned such that at least one portion of the sleeve external surface (220) is more distant from the nearest portion of the second sleeve external surface (280) than five times the bioreactor thickness (150) and at least one portion of the sleeve external surface (220) is less distant from the nearest portion of the second sleeve external surface (280) than five times the bioreactor thickness, and at least one portion of the third sleeve external surface (340) is more distant from the nearest portion of the second sleeve external surface (280) than five times the bioreactor thickness (150), and at least one portion of the third sleeve external surface (340) is less distant from the nearest portion of the sleeve external surface (280) than five times the bioreactor thickness (150).

In yet another embodiment, when in the presentation region (420), the sleeve (201), the second sleeve (260), and the third sleeve (320) are positioned such that at least one portion of the sleeve external surface (220) is more distant from the nearest portion of the second sleeve external surface (280) than two times the bioreactor thickness (150) and at least one portion of the sleeve external surface (220) is less distant from the nearest portion of the second sleeve external surface (280) than two times the bioreactor thickness, and at least one portion of the third sleeve external surface (340) is more distant from the nearest portion of the second sleeve external surface (280) than two times the bioreactor thickness (150), and at least one portion of the third sleeve external surface (340) is less distant from the nearest portion of the sleeve external surface (280) than two times the bioreactor thickness (150).

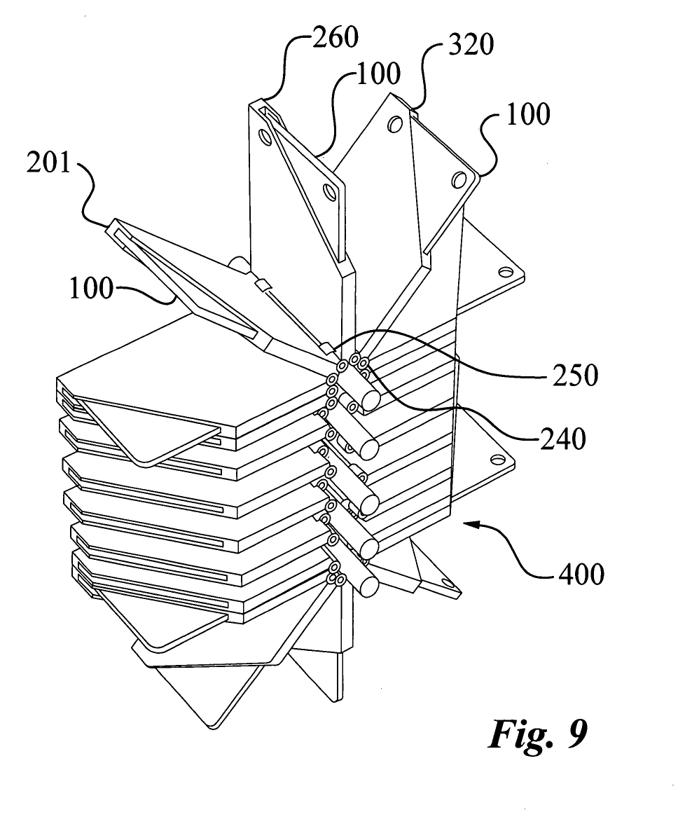

Thus, one skilled in the art will see that in one embodiment, seen in FIG. 16, the storage region (410) has a supine section (412) and a prone section (414), separated by the presentation region (420) and the bioreactor exterior surface (110) has a front (120) and a back (130). When the controlled environment bioreactor (100) is in the supine section (412), the bioreactor (100) is oriented so that gravity pulls the bioreactive material toward a back (130) of the bioreactor (100). Alternately, when the controlled environment bioreactor (100) is in the prone section (414), the bioreactor (100) is oriented so that gravity pulls the bioreactive material toward the front (120). The sleeve series (400) is movable through the supine section (412), the presentation region (420), and the prone section (414). This allows the contents of the controlled environment bioreactor (100) to agitate, and such movement tends to reverse the direction of gravitational force on any contents within the bioreactor (100) Further variations may be advantageous. In one embodiment, the sleeve series (400) further includes an inverted presentation region (430), seen well in FIGS. 9, 10, and 16, such that unidirectional motion of the sleeve series (400) moves the sleeve (201) from a position and return it to the same position of the sleeve series (400) moves the sleeve (201) through the presentation region (420), the prone section (414), the inverted presentation region (430), and the supine section (420). In other words, in this embodiment the inverted presentation region (430) creates a closed loop.

In one embodiment the sleeve series (400) consists of individual sleeves that are interconnected to form a chain-like sleeve series (400). As seen in FIG. 2, in this embodiment each sleeve (201) may include a leading coupling (240) and a following coupling (250), wherein the leading coupling (240) of one sleeve interconnects with the following coupling (250) of the adjacent sleeve, thereby creating a strong and flexible sleeve series (400).

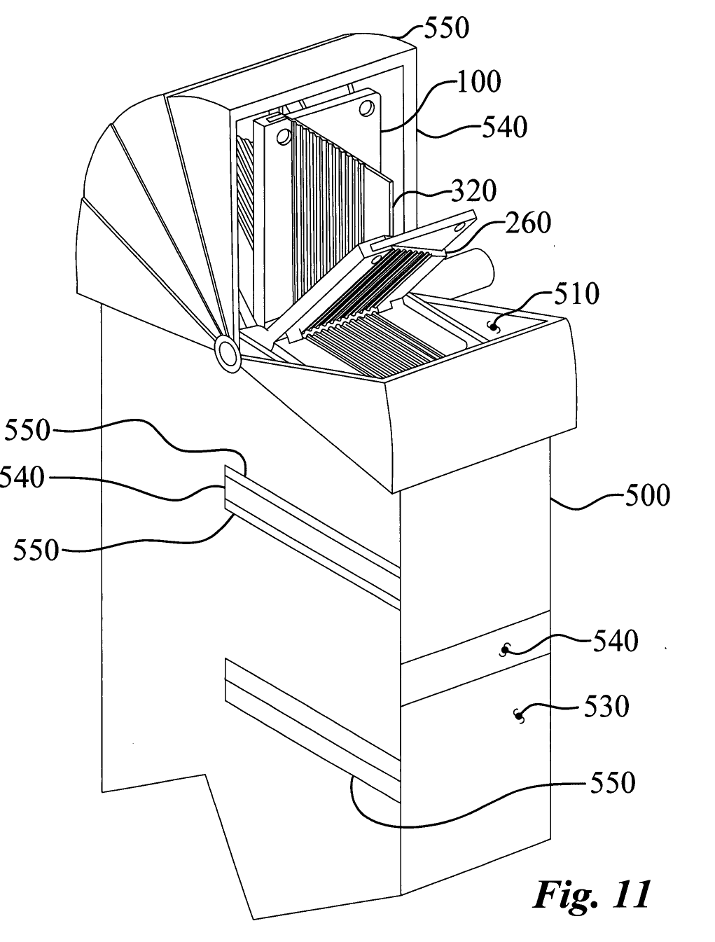

The sleeve series (400) may be surrounded in various manners. In one embodiment, the bioreactor analysis system (50) has a jacket (500), seen well in FIGS. 11-13, with a jacket interior surface (510), a jacket exterior surface (530), and a jacket interior surface (510) that forms an incubation chamber (520) that encloses the bioreactor (100) and the sleeve (201)) in a fluid at a fluid temperature. The jacket (500) may have an access port (540) that connects the jacket interior surface (510) with the jacket exterior surface (530). The jacket (500) may have a reversibly openable access shutter (550) cooperating with the access port (540) to reversibly occlude the access port (540). In such an embodiment, the access shutter (550) has an open position and a closed position, so that when the access shutter (550) is in the open position, the exposed portion (160) of the controlled environment bioreactor (100) is accessible to the mobile interventional assembly (810), and when the access shutter (550) is in the closed position, the access shutter (550) substantially prevents the fluid from passing through the access port (540). The access port (540) may be formed in the jacket (500) adjacent to the storage region (410), such that the exposed portion (160) of the controlled environment bioreactor (100) is accessible to the mobile interventional assembly (810) through the access port (540) while the sleeve (201) is in the storage region (410).

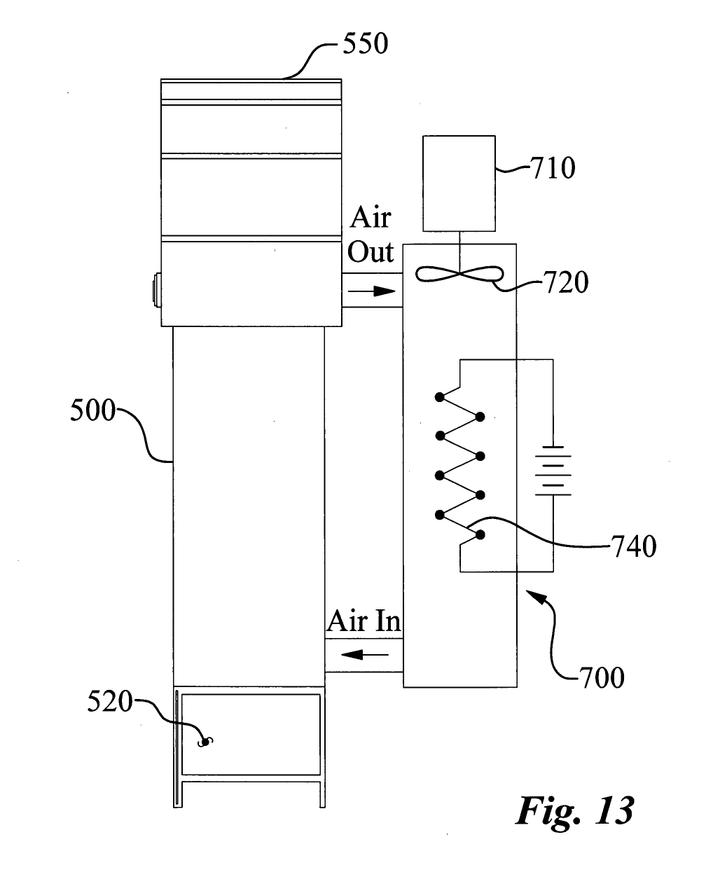

In another embodiment, the bioreactor analysis system (50) may have an incubation chamber temperature management system (700), seen schematically in FIG. 13, having a fluid temperature adjustment device (710), an energy source (740), and a fluid transfer means (730). The temperature adjustment device (710) and the fluid transfer means (720) are in fluid communication with the incubation chamber (520), so that when the temperature adjustment device (710) and the fluid transfer means (720) are energized, fluid circulates through the incubation chamber (520). Thus, the temperature adjustment device (710) controls the fluid temperature to substantially control a temperature in the bioreactor (100). The fluid path within the bioreactor analysis system (50) may encourage the flow of fluid within the sleeve thermal exchange channels (222).

In an alternate embodiment, the bioreactor analysis system (50) may have an incubation chamber temperature management system (700) having an energy source (740) that comprises a thermoelectric effect energy source. In yet another alternative embodiment, the bioreactor analysis system (50) may include an incubation chamber temperature management system (700) having an energy source (740) in heat transferable communication with the jacket (500). In such an embodiment, the fluid temperature adjustment device (710) controls the fluid temperature in the incubation chamber (520).

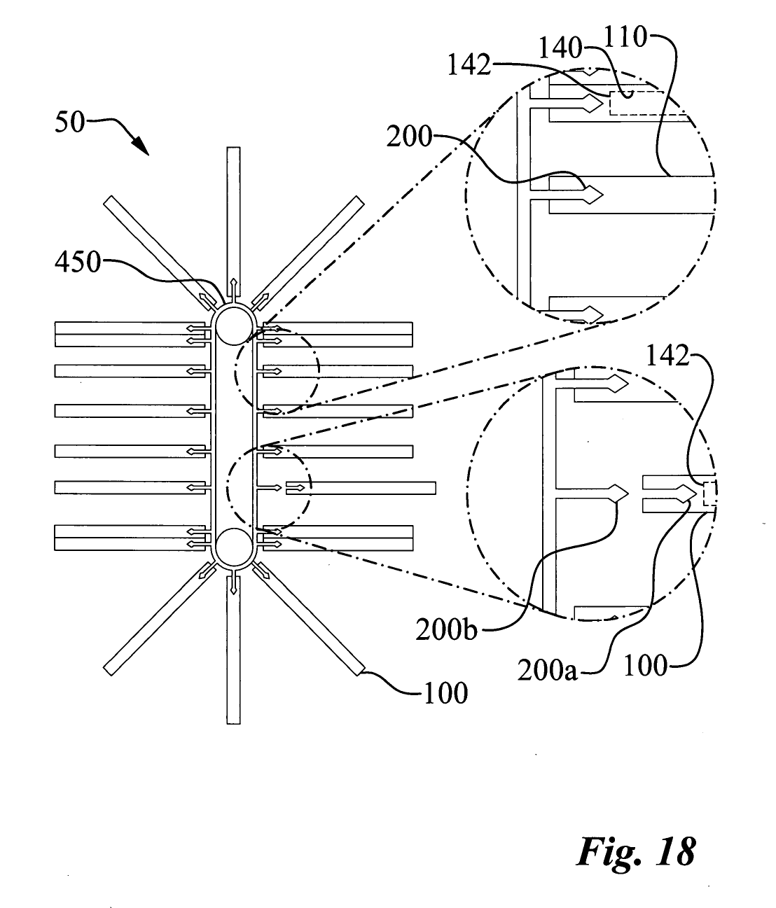

An embodiment of the bioreactor analysis system (50) may use means other than sleeves to contain one or more of the controlled environment bioreactors (100). In one illustrative embodiment, see in FIG. 18, a controlled environment bioreactor (100) has a bioreactor exterior surface (110) and a bioreactor interior surface (140), and the bioreactor interior surface (140) forms a chamber (142) for containing the bioreactive material.

There may be a storage array (450) releasably joinable to the controlled environment bioreactor (100), with joining means (200) for releasably joining the controlled environment bioreactor (100) and the storage array (450). One embodiment essentially incorporates a belt-type system storage array (450) to which the controlled environment bioreactor (100) releasably attach.

The bioreactor analysis system (50) may have an instrumentation system (800) having a mobile interventional assembly (810) for intervening with the bioreactive material, and the instrumentation system (800) may have a drive assembly (830) for moving the mobile interventional assembly (810). The drive assembly (830) may position the mobile interventional assembly (810) in interventional proximity to a portion of the controlled environment bioreactor (100), and the mobile interventional assembly (810) intervenes with the bioreactive material contained within the chamber (142) while the controlled environment bioreactor (100) remains releasably joined to the storage array (450).

One skilled in the art will understand that the joining means (200) may be of many different designs. In one embodiment, again seen in FIG. 18, the joining means (200) may be a bioreactor joining means (200a) formed in the controlled environment bioreactor (100) and configured to releasably cooperate with a storage array joining means (200b) formed in the storage array (450).

In an alternative embodiment, the joining means (200) may be a sleeve (201) having a sleeve interior surface (210) and a sleeve exterior surface (220), for releasably holding the controlled environment bioreactor (100). The sleeve interior surface (210) may form a slot (212) that slidably receives the controlled environment bioreactor (100) such that an exposed portion (160) of the controlled environment bioreactor (100) projects from the sleeve (201).

One skilled in the art will realize that many aspects of the instant invention are highly advantageous, even when used with non-controlled environment bioreactors (900), as opposed to controlled environment bioreactors (100). The term non-controlled environment bioreactors (900), as used herein, means a bioreactor (900) that is unable to self regulate at least one environmental parameter, such as humidity conservation or gas exchange. These bioreactors (900) must typically, but not always, be kept in external environments in which such variables as humidity and ambient gas mixture are controlled.

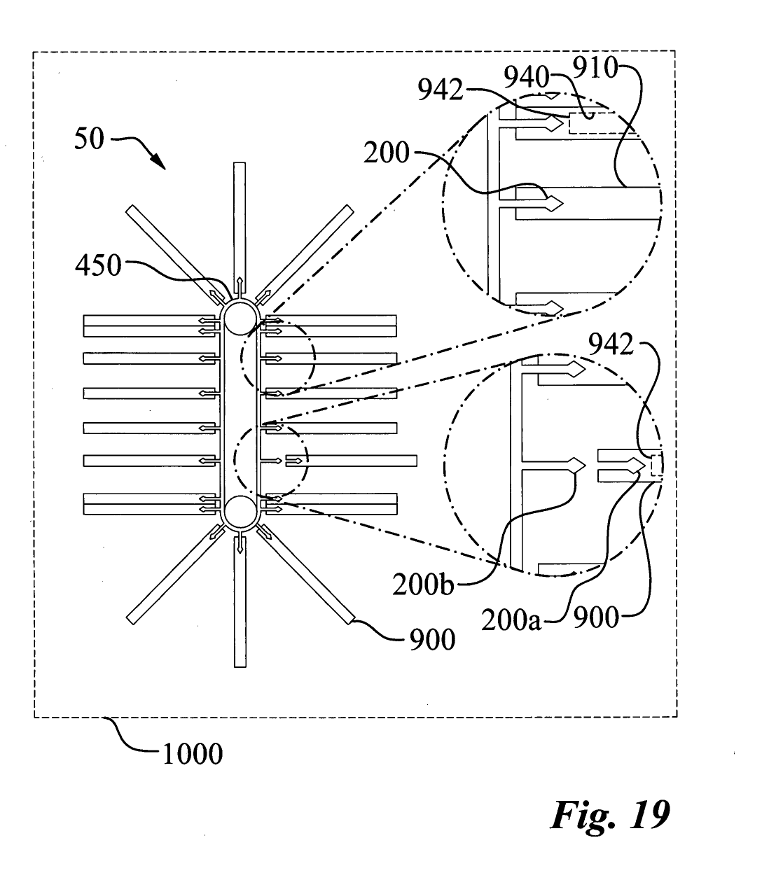

Therefore, in an alternative embodiment, as seen in FIG. 19, a bioreactor analysis system (50) for incubating and analysis of a bioreactive material includes a bioreactor (900) having a bioreactor exterior surface (910) and a bioreactor interior surface (940). The bioreactor interior surface (940) forms a chamber (942) for containing the bioreactive material.

There may be a storage array (450) releasably joinable to the controlled environment bioreactor (900); and joining means (200) for releasably joining the controlled environment bioreactor (900) and the storage array (450).

There also may be an instrumentation system (800) having a mobile interventional assembly (810) for intervening with the bioreactive material, with the instrumentation system (800) having a drive assembly (830) for moving the mobile interventional assembly (810). The drive assembly (830) may position the mobile interventional assembly (810) in interventional proximity to a portion of the bioreactor (900), and the mobile interventional assembly (810) intervenes with the bioreactive material contained within the chamber (942) while the controlled environment bioreactor (900) remains releasably joined to the storage array (450).

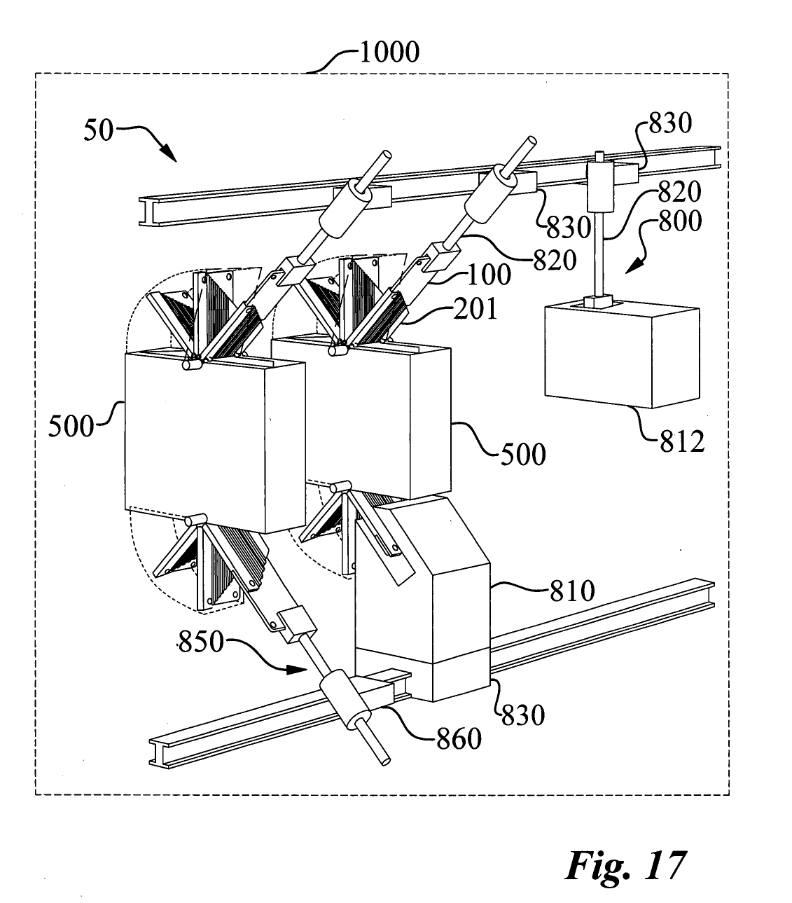

Typically, but not necessarily, in an embodiment utilizing non-controlled environment bioreactors, the bioreactor analysis system (50) may be enclosed within a biochamber (1000), similar to the biochamber seen surrounding the system in FIG. 17, providing controlled environmental conditions wherein the conditions are selected from the group consisting of temperature, humidity, atmospheric pressure, and ambient fluid composition.

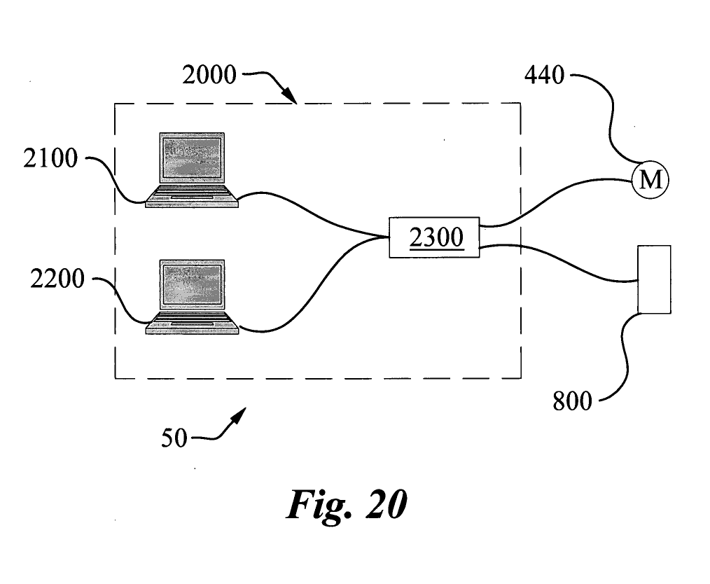

Any of the previously described embodiments of the bioreactor analysis system (50) may further include a control system (2000), shown schematically in FIG. 20, to control the operation of the sleeve drive (440) and the instrumentation system (800). The control system (2000) may include a first remote data input device (2100), a second remote data input device (2200), and a local data receiving device (2300). The local data receiving device (2300) is in operative communication with the sleeve drive (440), the instrumentation system (800), the first remote data input device (2100), and the second remote data input device (2200). The term “operative communication” includes, but is not limited to, wired and wireless data communication as would be known to one skilled in the art.

The first remote data input device (2100) receives a first sleeve control criteria from a first researcher consisting of a first set of control variables defining the operation and environment of the sleeve (201), and transmits the first sleeve control criteria to the local data receiving device (2300). The first remote data input device (2100) may be a personal computer, a personal digital assistant, or even a telephone, among other things. The first remote data input device (2100) may have software that prompts the first researcher to enter the requisite first set of control variables.

The first set of control variables may include, but is not limited to, rates and types of interventions such as microscopic examinations, centrifugations, and pH determination; and loading, removal, and replenishment of cells and media in the bioreactors.

The second remote data input device (2200) receives a second sleeve control criteria from a second researcher consisting of a first set of control variables defining the operation and environment of the second sleeve (260), and transmits the second sleeve control criteria to the local data receiving device (2300). The second remote data input device (2200) may be a personal computer, a personal digital assistant, or even a telephone, among other things. The second remote data input device (2200) may have software that prompts the second researcher to enter the requisite second set of control variables.

Like the first set of control variables, the second set of control variables may include, but is not limited to, rates and types of interventions such as microscopic examinations, centrifugations, and pH determination; and loading, removal, and replenishment of cells and media in the bioreactors.

In the present invention the location of the first remote data input device (2100), the second remote data input device (2200), and the local data receiving device (2300) are irrelevant. In fact, the control system (2000) is designed to allow researchers located in various parts of the world to control the bioreactor analysis system (50), also located anywhere, for a particular bioreactor (100) associated with the particular researcher. Therefore, as one skilled in the art will appreciate, an exemplary connected sleeve series (400) of the present invention may contain hundreds of sleeves with each sleeve containing a unique bioreactor assigned to a unique researcher that has remotely assigned control variables for their particular sleeve, or more appropriately their particular bioreactor.

The first sleeve control criteria and the second sleeve control criteria may be transmitted to the local data receiving device (2300) using any number of data transmission protocols. In one embodiment each remote data input device generates universal instruction code, similar to that of CNC machining code, that is transmitted to the local data receiving device (2300). In other embodiments, the sleeve control criteria may be transmitted simply as data representative of the control variables that is then transformed into machine instructions at the local data receiving device (2300).

The local data receiving device (2300) receives the first sleeve control criteria and the second sleeve control criteria and instructs the sleeve drive (440) and the instrumentation system (800) to perform the functions prescribed by the first sleeve control criteria and the second sleeve control criteria. The local data receiving device (2300) may be a personal computer or may simply be a relatively simple processor and standard memory modules, similar to that of a printer. The local data receiving device (2300) may be specifically allocated to a single sleeve series (400), or it may direct the operation of a number of sleeve series (400). Further, the local data receiving device (2300) may be an intelligent device that receives the control criteria and moves a particular bioreactor from one sleeve series to another if the control criteria necessitate conditions that may be inconsistent with the control criteria of other bioreactors within the initial sleeve series.

Numerous alterations, modifications, and variations of the embodiments disclosed herein will be apparent to those skilled in the art and they are all anticipated and contemplated to be within the spirit and scope of the instant invention. For example, although specific embodiments have been described in detail, those with skill in the art will understand that the preceding embodiments and variations can be modified to incorporate various types of substitute and or additional or alternative materials, relative arrangement of elements, and dimensional configurations. Accordingly, even though only few variations of the present invention are described herein, it is to be understood that the practice of such additional modifications and variations and the equivalents thereof, are within the spirit and scope of the invention as defined in the following claims. The corresponding structures, materials, acts, and equivalents of all means or step plus function elements in the claims below are intended to include any structure, material, or acts for performing the functions in combination with other claimed elements as specifically claimed.

I claim:

1. A bioreactor analysis system (50) for incubating and analysis of a bioreactive material, comprising:
a controlled environment bioreactor (100) having a bioreactor exterior surface (110), a bioreactor thickness (150), and a bioreactor interior surface (140), wherein the bioreactor interior surface (140) forms a chamber (142) for containing the bioreactive material;
a sleeve (201) having a sleeve interior surface (210) and a sleeve exterior surface (220), for releasably holding the controlled environment bioreactor (100), wherein the sleeve interior surface (210) forms a slot (212) that slidably receives the controlled environment bioreactor (100) such that an exposed portion (160) of the controlled environment bioreactor (100) projects from the sleeve (201); and
an instrumentation system (800) having a mobile interventional assembly (810) for intervening with the bioreactive material, and the instrumentation system (800) having a drive assembly (830) for moving the mobile interventional assembly (810), whereby the drive assembly (830) positions the mobile interventional assembly (810) in interventional proximity to the exposed portion (160) of the controlled environment bioreactor (100) that projects from the sleeve (201), and the mobile interventional assembly (810) intervenes with the bioreactive material contained within the chamber (142) while the controlled environment bioreactor (100) remains in the sleeve (201).

2. The bioreactor analysis system (50) of claim 1, wherein the instrumentation system (800) further includes a manipulator assembly (820) for intervening with the exposed portion (160) of the controlled environment bioreactor (100) projecting from the sleeve (201), whereby the drive assembly (830) positions the manipulator assembly (820) in releasable coupling proximity to the controlled environment bioreactor (100) when the controlled environment bioreactor (100) is in the sleeve (201);
the manipulator assembly (820) operable to releasably couple to the controlled environment bioreactor (100), move the controlled environment bioreactor (100) outside of the sleeve (201), and transport the controlled element bioreactor (100) to the fixed interventional assembly (812); and
the fixed interventional assembly (812) intervening with the bioreactive material contained within the chamber (142) while the controlled environment bioreactor (100) is outside the sleeve (201).

3. The bioreactor analysis system (50) of claim 1, wherein the sleeve exterior surface (220) further includes at least one thermal exchange channel (222) enhancing convection heat transfer between the sleeve (201) and an adjacent fluid.

4. The bioreactor analysis system (50) of claim 1, further including
(i) at least a second sleeve (260) adjacent to the sleeve (201) and the second sleeve (260) having a second sleeve exterior surface (280), and a third sleeve (320) adjacent to the second sleeve (260), the third sleeve (320) having a third sleeve exterior surface (340);
(ii) wherein the sleeve (201), the second sleeve (260), and the third sleeve (320) are arranged to form a connected sleeve series (400) having a storage region (410) and a presentation region (420), wherein in the presentation region (420) a primary sleeve exterior surface (221) of the sleeve is not parallel to a primary sleeve exterior surface (281) of the second sleeve (260) and is not parallel to a primary exterior surface (341) of the third sleeve (320);
(iii) a sleeve drive (440) positioned to move the sleeve series (400) through the storage region (410) and the presentation region (420); and
(iv) wherein the mobile interventional assembly (810) accesses the controlled environment bioreactor (100) while the controlled environment bioreactor (100) is in the presentation region (420).

5. The bioreactor analysis system (50) of claim 4, wherein in the storage region (410), the sleeve (201), the second sleeve (260), and the third sleeve (320) are positioned such that no portion of the sleeve external surface (220) is more distant from any portion of the second sleeve external surface (280) than ten times the bioreactor thickness (150), and no portion of the third sleeve external surface (340) is more distant from any portion of the second sleeve external surface (280) than ten times the bioreactor thickness (150).

6. The bioreactor analysis system (50) of claim 4, wherein in the storage region (410), the sleeve (201), the second sleeve (260), and the third sleeve (320) are positioned such that no portion of the sleeve external surface (220) is more distant from any portion of the second sleeve external surface (280) than five times the bioreactor thickness (150), and no portion of the third sleeve external surface (340) is more distant from any portion of the second sleeve external surface (280) than five times the bioreactor thickness (150).

7. The bioreactor analysis system (50) of claim 4, wherein in the storage region (410), the sleeve (201), the second sleeve (260), and the third sleeve (320) are positioned such that no portion of the sleeve external surface (220) is more distant from any portion of the second sleeve external surface (280) than two times the bioreactor thickness (150), and no portion of the third sleeve external surface (340) is more distant from any portion of the second sleeve external surface (280) than two times the bioreactor thickness (150);

8. The bioreactor analysis system (50) of claim 4, wherein when in the presentation region (420), the sleeve (201), the second sleeve (260), and the third sleeve (320) are positioned such that at least one portion of the sleeve external surface (220) is more distant from the nearest portion of the second sleeve external surface (280) than two times the bioreactor thickness (150), and at least one portion of the third sleeve external surface (340) is more distant from the nearest portion of the second sleeve external surface (280) than two times the bioreactor thickness (150).

9. The bioreactor analysis system (50) of claim 4, wherein when in the presentation region (420), the sleeve (201), the second sleeve (260), and the third sleeve (320) are positioned such that at least one portion of the sleeve external surface (220) is more distant from the nearest portion of the second sleeve external surface (280) than five times the bioreactor thickness (150), and at least one portion of the third sleeve external surface (340) is more distant from the nearest portion of the second sleeve external surface (280) than five times the bioreactor thickness (150).

10. The bioreactor analysis system (50) of claim 4, wherein when in the presentation region (420), the sleeve (201), the second sleeve (260), and the third sleeve (320) are positioned such that at least one portion of the sleeve external surface (220) is more distant from the nearest portion of the second sleeve external surface (280) than ten times the bioreactor thickness (150), and at least one portion of the third sleeve external surface (340) is more distant from the nearest portion of the second sleeve external surface (280) than ten times the bioreactor thickness (150).

11. The bioreactor analysis system (50) of claim 4, wherein when in the presentation region (420), the sleeve (201), the second sleeve (260), and the third sleeve (320) are positioned such that at least one portion of the sleeve external surface (220) is more distant from the nearest portion of the second sleeve external surface (280) than ten times the bioreactor thickness (150) and at least one portion of the sleeve external surface (220) is less distant from the nearest portion of the second sleeve external surface (280) than ten times the bioreactor thickness, and at least one portion of the third sleeve external surface (340) is more distant from the nearest portion of the second sleeve external surface (280) than ten times the bioreactor thickness (150), and at least one portion of the third sleeve external surface (340) is less distant from the nearest portion of the sleeve external surface (280) than ten times the bioreactor thickness (150).

12. The bioreactor analysis system (50) of claim 4, wherein when in the presentation region (420), the sleeve (201), the second sleeve (260), and the third sleeve (320) are positioned such that at least one portion of the sleeve external surface (220) is more distant from the nearest portion of the second sleeve external surface (280) than five times the bioreactor thickness (150) and at least one portion of the sleeve external surface (220) is less distant from the nearest portion of the second sleeve external surface (280) than five times the bioreactor thickness, and at least one portion of the third sleeve external surface (340) is more distant from the nearest portion of the second sleeve external surface (280) than five times the bioreactor thickness (150), and at least one portion of the third sleeve external surface (340) is less distant from the nearest portion of the sleeve external surface (280) than five times the bioreactor thickness (150).

13. The bioreactor analysis system (50) of claim 4, wherein when in the presentation region (420), the sleeve (201), the second sleeve (260), and the third sleeve (320) are positioned such that at least one portion of the sleeve external surface (220) is more distant from the nearest portion of the second sleeve external surface (280) than two times the bioreactor thickness (150) and at least one portion of the sleeve external surface (220) is less distant from the nearest portion of the second sleeve external surface (280) than two times the bioreactor thickness, and at least one portion of the third sleeve external surface (340) is more distant from the nearest portion of the second sleeve external surface (280) than two times the bioreactor thickness (150), and at least one portion of the third sleeve external surface (340) is less distant from the nearest portion of the sleeve external surface (280) than two times the bioreactor thickness (150).

14. The bioreactor analysis system (50) of claim 4, wherein the storage region (410) has a supine section (412) and a prone section (414), separated by the presentation region (420) and the bioreactor exterior surface (110) has a front (120) and a back (130), and wherein
(i) when the controlled environment bioreactor (100) is in the supine section (412), the bioreactor (100) is oriented so that gravity pulls the bioreactive material toward a back (130) of the bioreactor (100);
(ii) when the controlled environment bioreactor (100) is in the prone section (414), the bioreactor (100) is oriented so that gravity pulls the bioreactive material toward the front (120); and
(iii) the sleeve series (400) is movable through the supine section (412), the presentation region (420), and the prone section (414).

15. The bioreactor analysis system (50) of claim 14, wherein the sleeve series (400) further includes an inverted presentation region (430) such that unidirectional motion of the sleeve series (400) sufficient to move the sleeve (201) from a position and return it to the same position of the sleeve series (400) moves the sleeve (201) through the presentation region (420), the prone section (414), the inverted presentation region (430), and the supine section (420).

16. The bioreactor analysis system (50) of claim 1, further including
a jacket (500) having a jacket interior surface (510), a jacket exterior surface (530), and an a jacket interior surface (510) forms an incubation chamber (520) that encloses the bioreactor (100) and the sleeve (201)) in a fluid at a fluid temperature; and
(ii) an access port (540) connects the jacket interior surface (510) with the jacket exterior surface (530).

17. The bioreactor analysis system (50) of claim 16, wherein the jacket (500) further includes a reversibly openable access shutter (550) cooperating with the access port (540) to reversibly occlude the access port (540), wherein
the access shutter (550) has an open position and a closed position, whereby when the access shutter (550) is in the open position, the exposed portion (160) of the controlled environment bioreactor (100) is accessible to the mobile interventional assembly (810), and
when the access shutter (550) is in the closed position, the access shutter (550) substantially prevents the fluid from passing through the access port (540).

18. The bioreactor analysis system of claim 16, wherein the access port (540) is formed in the jacket (500) adjacent to the storage region (410), such that the exposed portion (160) of the controlled environment bioreactor (100) is accessible to the mobile interventional assembly (810) through the access port (540) while the sleeve (201) is in the storage region (410)

19. The bioreactor analysis system (50) of claim 16, further including an incubation chamber temperature management system (700) having a fluid temperature adjustment device (710), and energy source (740), and a fluid transfer means (730), wherein the temperature adjustment device (710) and the fluid transfer means (720) are in fluid communication with the incubation chamber (520), whereby when the temperature adjustment device (710) and the fluid transfer means (720) are energized, fluid circulates through the incubation chamber (520) and the temperature adjustment device (710) controls the fluid temperature to substantially control a temperature in the bioreactor (100).

20. The bioreactor analysis system (50) of claim 16, further including an incubation chamber temperature management system (700) having an energy source (740) further comprising a thermoelectric effect energy source.

21. The bioreactor analysis system (50) of claim 16, further including an incubation chamber temperature management system (700) having an energy source (740) in heat transferable communication with the jacket (500), whereby the fluid temperature adjustment device (710) controls the fluid temperature in the incubation chamber (520).

22. A bioreactor analysis system (50) for incubating and analysis of a bioreactive material, comprising:
a controlled environment bioreactor (100) having a bioreactor exterior surface (110) and a bioreactor interior surface (140), wherein the bioreactor interior surface (140) forms a chamber (142) for containing the bioreactive material;
a storage array (450) releasably joinable to the controlled environment bioreactor (100);
joining means (200) for releasably joining the controlled environment bioreactor (100) and the storage array (450), and;
an instrumentation system (800) having a mobile interventional assembly (810) for intervening with the bioreactive material, and the instrumentation system (800) having a drive assembly (830) for moving the mobile interventional assembly (810), whereby the drive assembly (830) positions the mobile interventional assembly (810) in interventional proximity to a portion of the controlled environment bioreactor (100), and the mobile interventional assembly (810) intervening with the bioreactive material contained within the chamber (142) while the controlled environment bioreactor (100) remains releasably joined to the storage array (450).

23. The bioreactor analysis system (50) of claim 22, wherein the joining means (200) is a bioreactor joining means (200a) formed in the controlled environment bioreactor (100) and configured to releasably cooperate with a storage array joining means (200b) formed in the storage array (450).

24. The bioreactor analysis system (50) of claim 22, wherein the joining means (200) is a sleeve (201) having a sleeve interior surface (210) and a sleeve exterior surface (220), for releasably holding the controlled environment bioreactor (100), wherein the sleeve interior surface (210) forms a slot (212) that slidably receives the controlled environment bioreactor (100) such that an exposed portion (160) of the controlled environment bioreactor (100) projects from the sleeve (201).

25. A bioreactor analysis system (50) for incubating and analysis of a bioreactive material, comprising:
a bioreactor (900) having a bioreactor exterior surface (910) and a bioreactor interior surface (940), wherein the bioreactor interior surface (940) forms a chamber (942) for containing the bioreactive material;
a storage array (450) releasably joinable to the controlled environment bioreactor (900);
joining means (200) for releasably joining the controlled environment bioreactor (900) and the storage array (450), and;
an instrumentation system (800) having a mobile interventional assembly (810) for intervening with the bioreactive material, and the instrumentation system (800) having a drive assembly (830) for moving the mobile interventional assembly (810), whereby the drive assembly (830) positions the mobile interventional assembly (810) in interventional proximity to a portion of the bioreactor (900), and the mobile interventional assembly (810) intervening with the bioreactive material contained within the chamber (942) while the controlled environment bioreactor (900) remains releasably joined to the storage array (450).

26. The bioreactor analysis system (50) of claim 25, wherein the bioreactor analysis system (50) is enclosed within a biochamber (1000), providing controlled environmental conditions wherein the conditions are selected from the group consisting of temperature, humidity, atmospheric pressure, and ambient fluid composition.

27. A bioreactor analysis system (50) for incubating and analysis of a bioreactive material, comprising:
a controlled environment bioreactor (100) having a bioreactor exterior surface (110), a bioreactor thickness (150) and a bioreactor interior surface (140), wherein the bioreactor interior surface (140) forms a chamber (142) for containing the bioreactive material;
a sleeve (201) having a sleeve interior surface (210) and a sleeve exterior surface (220), for releasably holding the controlled environment bioreactor (100), wherein the sleeve interior surface (210) forms a slot (212) that slidably receives the controlled environment bioreactor (100) such that an exposed portion (160) of the controlled environment bioreactor (100) projects from the sleeve (201);
a fixed interventional assembly (812) in a predetermined fixed position;
a manipulator assembly (820) for intervening with the exposed portion (160) of the controlled environment bioreactor (100) projecting from the sleeve (201), whereby the drive assembly (830) positions the manipulator assembly (820) in releasable coupling proximity to the controlled environment bioreactor (100) when the controlled environment bioreactor (100) is in the sleeve (201);
the manipulator assembly (820) operable to releasably couple to the controlled environment bioreactor (100), move the controlled environment bioreactor (100) outside of the sleeve (201), and transport the controlled element bioreactor (100) to the fixed interventional assembly (812)
the fixed interventional assembly (812) intervening with the bioreactive material contained within the chamber (142) while the controlled environment bioreactor (100) is outside the sleeve (201).

28. A bioreactor analysis system (50) for incubating and analysis of a bioreactive material, comprising:
a controlled environment bioreactor (100) having a bioreactor exterior surface (110), a bioreactor thickness (150), and a bioreactor interior surface (140), wherein the bioreactor interior surface (140) forms a chamber (142) for containing the bioreactive material;
a sleeve (201) having a sleeve interior surface (210) and a sleeve exterior surface (220), for releasably holding the controlled environment bioreactor (100), wherein the sleeve interior surface (210) forms a slot (212) that slidably receives the controlled environment bioreactor (100) such that an exposed portion (160) of the controlled environment bioreactor (100) projects from the sleeve (201);
a second sleeve (260) adjacent to the sleeve (201), wherein the second sleeve (260) has a second sleeve exterior surface (280);
a third sleeve (320) adjacent to the second sleeve (260), wherein the third sleeve (320) has a third sleeve exterior surface (340);
a connected sleeve series (400) formed of the sleeve (201), the second sleeve (260), and the third sleeve (320), wherein the connected sleeve series (400) has a storage region (410) and a presentation region (420);
a sleeve drive (440) positioned to move the sleeve series (400) through the storage region (410) and the presentation region (420);
an instrumentation system (800) having a mobile interventional assembly (810) for interacting with the bioreactive material, and the instrumentation system (800) having a drive assembly (830) for moving the mobile interventional assembly (810), whereby the drive assembly (830) positions the mobile interventional assembly (810) in interventional proximity to the exposed portion (160) of the controlled environment bioreactor (100) that projects from the sleeve (201), and the mobile interventional assembly (810) interacts with the bioreactive material contained within the chamber (142) while the controlled environment bioreactor (100) remains in the sleeve (201); and
a control system (2000) including a first remote data input device (2100), a second remote data input device (2200), and a local data receiving device (2300), wherein:
(i) the local data receiving device (2300) is in operative communication with the sleeve drive (440), the instrumentation system (800), the first remote data input device (2100), and the second remote data input device (2200);
(ii) the first remote data input device (2100) receives a first sleeve control criteria from a first researcher consisting of a first set of control variables defining the operation and environment of the sleeve (201), and transmits the first sleeve control criteria to the local data receiving device (2300);
(iii) the second remote data input device (2200) receives a second sleeve control criteria from a second researcher consisting of a second set of control variables defining the operation and environment of the second sleeve (260), and transmits the second sleeve control criteria to the local data receiving device (2300); and
(iv) the local data receiving device (2300) receives the first sleeve control criteria and the second sleeve control criteria and instructs the sleeve drive (440) and the instrumentation system (800) to perform the functions prescribed by the first sleeve control criteria and the second sleeve control criteria.
